(19) United States
(12) Patent Application Publication
(10) Pub. No.: US 2024/0138906 A1
(43) Pub. Date: May 2, 2024

(54) Alignment Aid for Ablation Procedures

(71) Applicant: BIOSENSE WEBSTER (ISRAEL) LTD., Yokneam (IL)

(72) Inventors: Naor Matalon, Haifa (IL); Lidya Tonkonogi Tarasenko, Haifa (IL); Sapir Adam, Malot (IL)

(21) Appl. No.: 18/369,974

(22) Filed: Sep. 19, 2023

Related U.S. Application Data
(60) Provisional application No. 63/420,139, filed on Oct. 28, 2022.

(51) Int. Cl. A61B 18/14; A61B 90/00

(52) U.S. Cl. CPC A61B 18/1492; A61B 90/37; A61B 2018/00577

(57) ABSTRACT

A method, consisting of registering a multiplicity of locations of ablated sites of an organ of a human subject. The method includes manipulating a catheter having a plurality of electrodes in proximity to the organ, the electrodes being configured to further ablate selected sites of the organ. The method further includes rendering on a display an organ icon showing the organ and the multiplicity of the locations of the ablated sites, and a catheter icon showing the plurality of electrodes, and providing an indication on the display of a quality of alignment between the plurality of electrodes and the multiplicity of the locations of the ablated sites.

### CROSS-REFERENCE TO RELATED APPLICATION

This application claims the benefit of U.S. Provisional Patent Application 63/420,139, filed Oct. 28, 2022, which is incorporated herein by reference.

### FIELD OF THE DISCLOSURE

The present disclosure is related generally to ablation, and specifically to ablation of an organ of a human subject.

### BACKGROUND

To ablate a region of an organ of a human subject, such as a portion of the heart, a probe having means for ablation may be navigated to the region. As it is navigated, the probe may be tracked, for example by an electromagnetic tracking system, to ensure that it has reached the desired region. However, in cases where the probe is inserted through a vein or artery of the subject, the flexibility of the probe, as well as the typically tortuous path taken by the probe, mean that maneuvering the probe to reach the desired region is not trivial.

### BRIEF DESCRIPTION OF THE DRAWINGS

FIG. 1 is a schematic illustration of a catheter-based electrophysiology mapping and ablation system;

FIG. 2 is a flowchart of steps followed during an ablation procedure performed with the system of FIG. 1; and

FIGS. 3A-3D and FIGS. 4A-4B are schematic figures illustrating the steps.

### DESCRIPTION OF EXAMPLES

### Overview

In an ablation procedure on the ostium of a pulmonary vein (PV), the object is to prevent transference of electrical signals between the PV and the heart chamber to which the vein is connected. The object may be achieved by a professional performing a series of ablations in a closed circumferential line on the ostium, so that there are no gaps in the line, and so that the line lies approximately in a plane circling the ostium. In many cases, a “first ablation pass” does leave some gaps, so that it is necessary to repeat the ablation to seal the gaps.

This may be the case even with a catheter that is designed to assist in the production of the closed circumferential line. Such a catheter is a “lasso” catheter, which the professional may position in an ostium so that its electrodes lie on an arc. After an initial set of ablations is performed with the catheter, the lasso may be rotated so that in a new position the new arc of the electrodes is in the initial plane and completes the line around the ostium, so that ablating with the newly positioned electrodes should form a set of ablation points with no gaps.

However, it is not a trivial task for the professional to maneuver and align the catheter as is described above, or to keep it stable while the ablations are performed. Consequently, there are a number of ablation procedures where gaps are generated, and these gaps need to be sealed.

Examples of the present disclosure assist the professional by presenting on a display an indication of a gap in ablations that have already been performed. An icon showing the position and orientation of the ablation catheter is also shown, together with an indication on the icon, as at least one number and/or at least one color, that assist the physician in aligning the catheter with existing ablation sites, and so that it is in the plane of the sites. Once aligned, the lasso catheter may be rotated in the plane so that electrodes of the catheter are in the gap, and these electrodes may then be activated to ablate respective sections of the ostium to seal the gap.

### System Description

In the following description, like elements are identified by the same numeral, and are differentiated, where required, by having a letter attached as a suffix to the numeral.

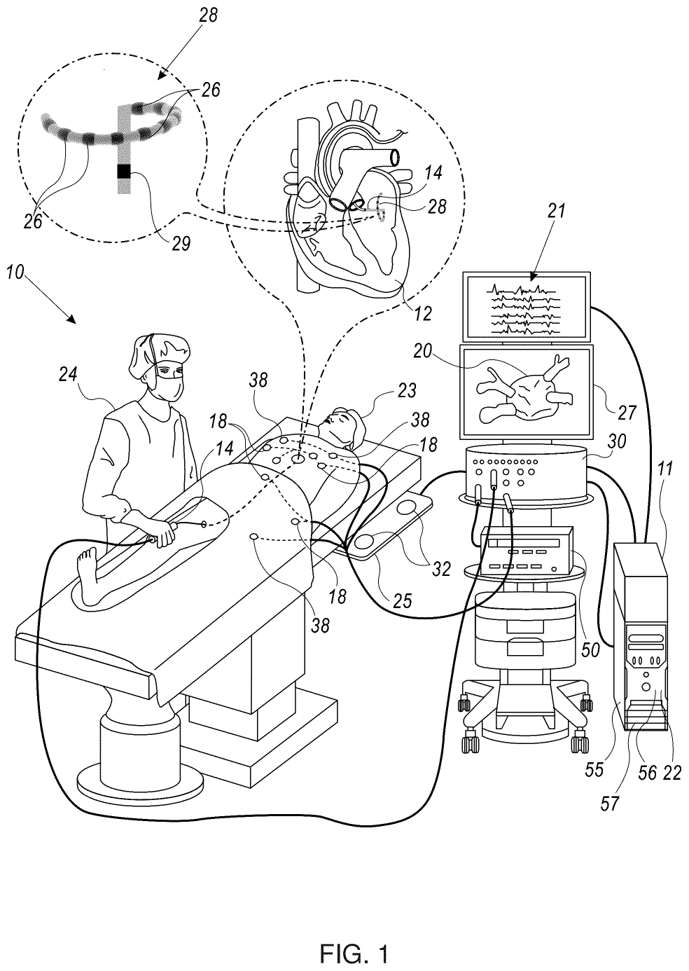

Reference is now made to FIG. 1 which shows a catheter-based electrophysiology mapping and ablation system 10. System 10 includes multiple catheters, which are percutaneously inserted by a physician 24 through the patient's vascular system into a chamber or vascular structure of a heart 12. Typically, a delivery sheath catheter is inserted into the left or right atrium near a desired location in heart 12. Thereafter, a plurality of catheters can be inserted into the delivery sheath catheter so as to arrive at the desired location. The plurality of catheters may include catheters dedicated for sensing Intracardiac Electrogram (IEGM) signals, catheters dedicated for ablating and/or catheters dedicated for both sensing and ablating. An example catheter 14 that is configured for ablation is illustrated herein. Physician 24 brings a distal tip 28 of catheter 14 into contact with the heart wall for ablating a target site in heart 12.

Catheter 14 is an exemplary lasso catheter that includes multiple electrodes 26 distributed around distal tip 28. Catheter 14 may additionally include a position sensor 29 embedded in or near distal tip 28 for tracking the position and orientation of distal tip 28. Optionally, position sensor 29 is a magnetic based position sensor including three magnetic coils for sensing three-dimensional (3D) position and orientation.

Magnetic based position sensor 29 may be operated together with a location pad 25 including a plurality of magnetic coils 32 configured to generate magnetic fields in a predefined working volume. The real time position of distal tip 28 of catheter 14 may be tracked based on magnetic fields generated with location pad 25 and sensed by magnetic based position sensor 29. Details of the magnetic based position sensing technology are described in U.S. Pat. Nos. 5,5391,199; 5,443,489; 5,558,091; 6,172,499; 6,239,724; 6,332,089; 6,484,118; 6,618,612; 6,690,963; 6,788,967; 6,892,091.

System 10 includes one or more electrode patches 38 positioned for skin contact on a patient 23 to establish location reference for location pad 25 as well as impedance-based tracking of electrodes 26. For impedance-based tracking, electrical current is directed toward electrodes 26 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182.

A recorder 11 displays electrograms 21 captured with body surface ECG electrodes 18 and intracardiac electrograms (IEGM) that may be captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more of electrodes 26. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between catheters, electrophysiological equipment, a power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, a processor 22 with memory or storage with appropriate operating software loaded therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of distal tip 28 within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

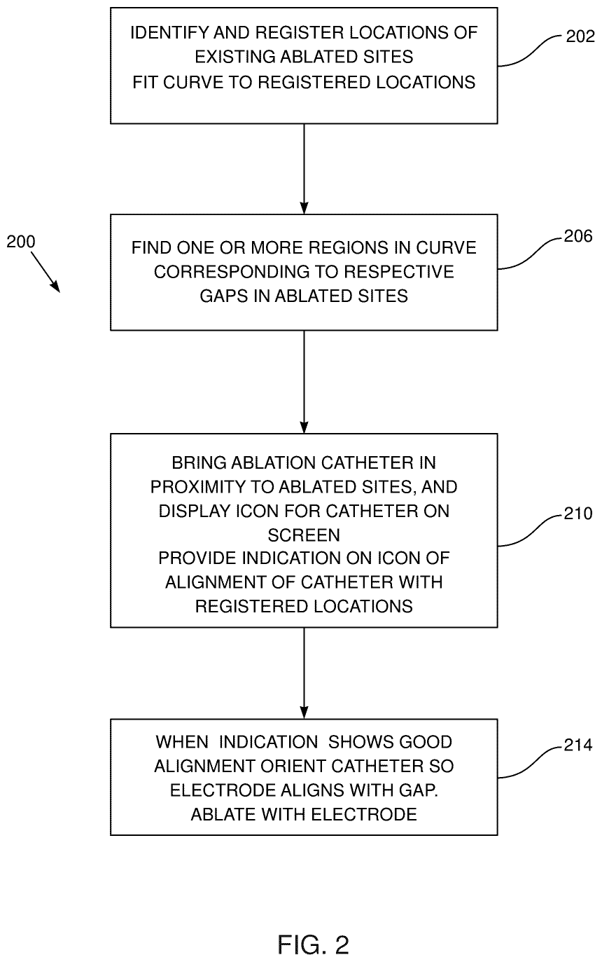

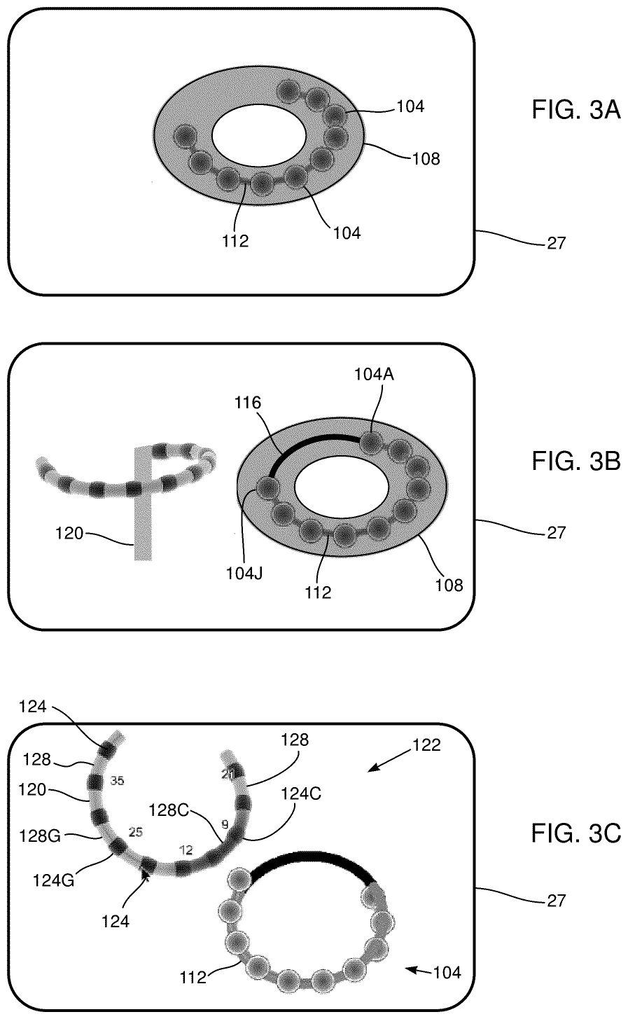

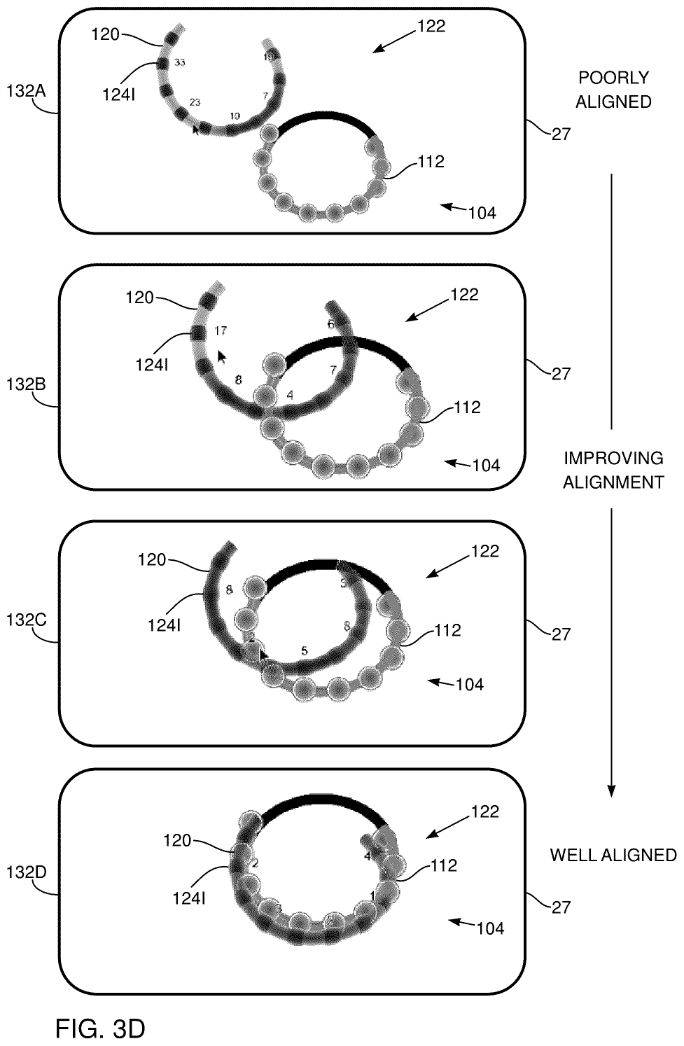

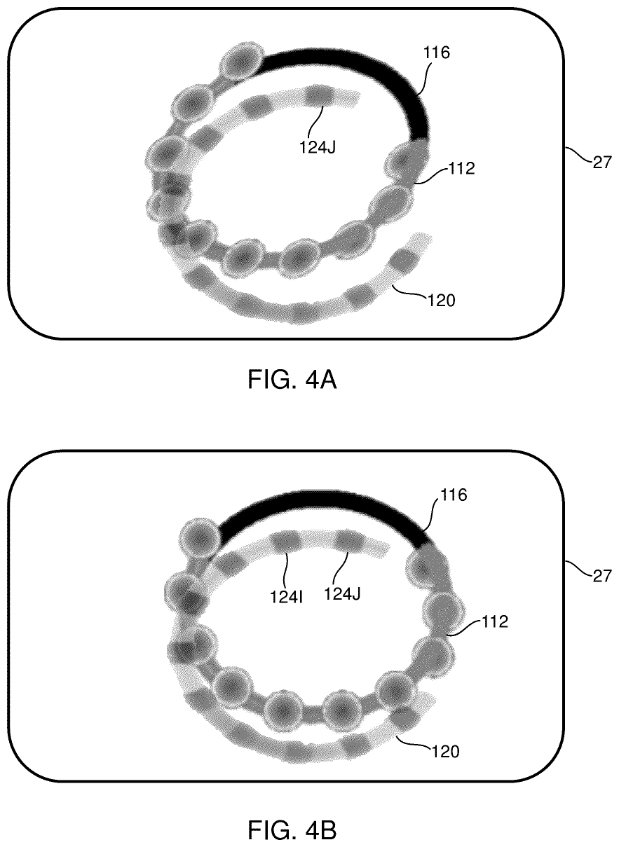

FIG. 2 is a flowchart 200 of steps followed by processor 22 and physician 24 during an ablation procedure performed with system 10, and FIGS. 3A-3D and FIGS. 4A-4B are schematic figures illustrating the steps.

In an initial step 202 of the flowchart, it is assumed that system 10 has already been used to ablate an organ of patient 23, herein by way of example assumed to comprise an ostium of a pulmonary vein (PV) of patient 23, and symbols 104 depicting the locations of the initial ablation sites are displayed on a representation 108 of the example organ, the PV, that is presented on device 27, as shown in FIG. 3A. Representation 108 is also herein termed pulmonary vein icon 108. From locations of symbols 104 that are recorded during their respective ablations, processor 22, uses interpolation to fit a curve 112 to the ablated symbols, and this may also be displayed on device 27.

In an analysis step 206 processor 22 examines the recorded ablation symbol locations, looking for gaps between the symbols. In an example, a gap is assumed to occur if the separation between adjacent ablation symbol locations is greater than a preset value, herein assumed to be 5 mm. However, the preset value may be more or less than 5 mm. Processor 22 may indicate the presence of a gap, and its location, by coloring an appropriate section of curve 112 with a different color from that used in the initial step.

Furthermore, since the curve joining ablation symbols of the ostium should be a closed curve, a gap may also be indicated by adding an arc to the interpolated curve of step 202 so as to close the curve. As is illustrated schematically in FIG. 3B, processor 22 has added such an arc, arc 116, to curve 112, the arc indicating that there is a gap between symbols 104A and 104J.

Processor 22 may determine that there is more than one gap between symbols 104, in which case for the following steps of the flowchart physician 24 may be prompted to select the gap to be closed. In the following description the gap to be closed is assumed to comprise the gap represented by arc 116, and those having ordinary skill in the art will be able to adapt the description, mutatis mutandis, for other gaps.

In a manipulation step 210 physician 24 manipulates lasso catheter 14 in proximity to the ablation site locations. As catheter 14 is manipulated, processor 22 records the position and orientation of the catheter, and the processor uses the recorded values to position and orient a catheter icon 120 in the vicinity of PV icon 108 and symbols 104 on device 27, as is also illustrated schematically in FIG. 3B. The presentation of catheter icon 120 and PV icon 108 with symbols 104 assists physician 24 in navigating catheter 14 to a desired region of the PV, such as to the gap illustrated by arc 116.

As is stated above, it is not trivial to correctly manipulate the lasso catheter to a desired region. In an example of the disclosure, an indication 122 is provided for catheter icon 120 that assists physician 24 in aligning the electrodes of the catheter with the existing ablation sites. (Once well aligned, in a subsequent manipulation step 214 described below, physician 24 may rotate the lasso catheter so that at least one of its electrodes aligns with the gap.)

The indication 122 may be alphanumerical and/or graphical. In an example of the disclosure illustrated in FIG. 3C, the indication comprises a numerical value of the distance for selected electrodes 26 of catheter 14 to curve 112. The numerical value is positioned in proximity to respective representations 124 of the selected electrodes on icon 120. Thus, as illustrated in FIG. 3C, representation 124G indicates that its electrode 26 is a distance of 25 mm from curve 112, and representation 124C indicates that its electrode is 9 mm from the curve, so that the electrode of representation 124C is closer to the curve than the electrode of representation 124G.

In an example of the disclosure also illustrated in FIG. 3C, the indication 122 also comprises a color change, between a first color indicating close proximity and a second color indicating more distant proximity. In a disclosed example the first color is green and the second color is red, but it will be understood that in other examples other colors may be used. In the disclosed example sections 128 of icon 120 between representations 124 are colored 100% red, except for sections that are within a preset distance from curve 112, which are colored a mixture of red and green, and are 100% green for a closer distance. In one example of the disclosure the preset distance is 15 mm, and the closer distance is 5 mm, but in other examples the values of the preset and closer distances are different from 15 mm and 5 mm. In the figures, the increase in green is illustrated by using a darker shade of gray. Thus, as illustrated in FIG. 3C, a section 128G adjacent to representation 124G is red, whereas a section 128C adjacent to representation 124C is green.

FIG. 3D is a set of schematic illustrations 132 of display device 27 as catheter 14 is manipulated to improve its alignment with the initial ablation sites, depicted by symbols 104 on curve 112. Illustration 132A shows there is poor alignment between the lasso catheter and the existing ablation sites; for example the distance of the electrode having representation 124I is large, 33, and the surrounding region is red. As is indicated in illustrations 132B and 132C the distance reduces, to 17 and then to 8, and the surrounding region becomes green. Illustration 132D depicts catheter 14 as being well aligned with the ablation sites, since the distance numbers of all the electrodes have low value.

In the well aligned state depicted in illustration 132D the positions of the electrodes of catheter 14 are close to the positions of the initial ablation sites. Following from this, a plane encompassing the electrodes of catheter 14 and a plane encompassing the initial ablation sites have a common orientation.

In subsequent manipulation step 214, once physician 24 has manipulated catheter 14 so that it has a common orientation with the initial ablation sites, as depicted in illustration 132D, the physician may re-orient the catheter so that at least one electrode covers the ablation site gap depicted by curve 116. The re-orientation is herein assumed to comprise physician 24 rotating catheter 14 in its plane, so that at least one electrode of the catheter aligns with the identified gap, and processor depicts the rotations on device 27.

Two examples of rotations are illustrated in FIGS. 4A and 4B. In both figures catheter icon 120, which would normally overlay arc 116 and curve 112, is for clarity shown displaced vertically downwards from its actual position.

FIG. 4A illustrates that catheter 14 has been rotated so that the electrode depicted by representation 124J covers arc 116. FIG. 4B illustrates that catheter 14 has been rotated further, so that the electrodes depicted by representations 124I and 124J cover arc 116.

Once catheter 14 has been rotated, as exemplified by FIG. 4A or FIG. 4B, physician 24 may activate the appropriate electrodes to ablate regions of curve 116 covered by the electrodes.

It will be understood that the steps of 200 may be reiterated, as necessary, to ensure that there is no remaining gap between ablation sites.

### EXAMPLES

Example 1: A method, comprising:
registering a multiplicity of locations of ablated sites (104) of an organ of a human subject;
manipulating a catheter (14) having a plurality of electrodes (26) in proximity to the organ, the electrodes being configured to further ablate selected sites of the organ; and
rendering on a display (27) an organ icon (108) showing the organ and the multiplicity of the locations of the ablated sites, and a catheter icon (120) showing the plurality of electrodes, and providing an indication (122) on the display of a quality of alignment between the plurality of electrodes and the multiplicity of the locations of the ablated sites.

Example 2: The method according to example 1, and comprising identifying a gap in the locations, and, when the indication denotes there is good alignment, further manipulating the catheter so that at least one of the plurality of electrodes aligns with a section of the gap, and activating the at least one electrode to ablate the section.

Example 3: The method according to example 2, wherein the multiplicity of the locations of the ablated sites define an ablation site plane, and the plurality of the electrodes define an electrode plane, the method further comprising that a position and an orientation of the ablation site plane and the electrode plane correspond for the good alignment.

Example 4: The method according to example 2, wherein identifying the gap comprises identifying from amongst the registered locations adjacent locations that are separated by greater than a preset distance.

Example 5: The method according to example 2, wherein the organ comprises a pulmonary vein of the human subject, and wherein identifying the gap comprises fitting a closed curve to the locations of the ablated sites, and indicating on the display at least one site of the closed curve wherein adjacent locations are separated by a distance greater than a preset threshold distance.

Example 6: The method according to example 1, wherein the plurality of electrodes are configured to further ablate using at least one of a radiofrequency current and irreversible electroporation.

Example 7: The method according to example 1, wherein the catheter comprises a lasso catheter.

Example 8: The method according to example 1, wherein the indication comprises at least one of respective numerical values of distances between the plurality of the electrodes and the multiplicity of the locations and a color indicative of the values of the distances.

Example 9: Apparatus, comprising:
a display device (27);
a catheter (14) having a plurality of electrodes (26) configured to ablate selected sites of an organ of a human subject; and
a processor (22), configured to:
register a multiplicity of locations of ablated sites of the organ;
record manipulations of the catheter in proximity to the organ;
render on the display device an organ icon (108) showing the organ and the multiplicity of the locations of the ablated sites, and a catheter icon (120) showing the plurality of electrodes; and
provide an indication (122) on the display device of a quality of alignment between the plurality of electrodes and the multiplicity of the locations of the ablated sites.

Example 10: The apparatus according to example 9, and comprising the processor being configured to:
identify a gap in the locations, and, when the indication denotes there is good alignment,
record further manipulations of the catheter so that at least one of the plurality of electrodes aligns with a section of the gap, and
activate the at least one electrode to ablate the section.

Example 11: The apparatus according to example 10, wherein the multiplicity of the locations of the ablated sites define an ablation site plane, and the plurality of the electrodes define an electrode plane, so that a position and an orientation of the ablation site plane and the electrode plane correspond for the good alignment.

Example 12: The apparatus according to example 10, wherein identifying the gap comprises identifying from amongst the registered locations adjacent locations that are separated by greater than a preset distance.

Example 13: The apparatus according to example 10, wherein the organ comprises a pulmonary vein of the human subject, and wherein identifying the gap comprises fitting a closed curve to the locations of the ablated sites, and indicating on the display device at least one site of the closed curve wherein adjacent locations are separated by a distance greater than a preset threshold distance.

Example 14: The apparatus according to example 9, wherein the plurality of electrodes are configured to ablate using at least one of a radiofrequency current and irreversible electroporation.

Example 15: The apparatus according to claim 9, wherein the catheter comprises a lasso catheter.

Example 16: The apparatus according to example 9, wherein the indication comprises at least one of respective numerical values of distances between the plurality of the electrodes and the multiplicity of the locations and a color indicative of the values of the distances.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method, comprising:
registering a multiplicity of locations of ablated sites of an organ of a human subject;
manipulating a catheter having a plurality of electrodes in proximity to the organ, the electrodes being configured to further ablate selected sites of the organ; and
rendering on a display an organ icon showing the organ and the multiplicity of the locations of the ablated sites, and a catheter icon showing the plurality of electrodes, and providing an indication on the display of a quality of alignment between the plurality of electrodes and the multiplicity of the locations of the ablated sites.

2. The method according to claim 1, and comprising identifying a gap in the locations, and, when the indication denotes there is good alignment, further manipulating the catheter so that at least one of the plurality of electrodes aligns with a section of the gap, and activating the at least one electrode to ablate the section.

3. The method according to claim 2, wherein the multiplicity of the locations of the ablated sites define an ablation site plane, and the plurality of the electrodes define an electrode plane, the method further comprising that a position and an orientation of the ablation site plane and the electrode plane correspond for the good alignment.

4. The method according to claim 2, wherein identifying the gap comprises identifying from amongst the registered locations adjacent locations that are separated by greater than a preset distance.

5. The method according to claim 2, wherein the organ comprises a pulmonary vein of the human subject, and wherein identifying the gap comprises fitting a closed curve to the locations of the ablated sites, and indicating on the display at least one site of the closed curve wherein adjacent locations are separated by a distance greater than a preset threshold distance.

6. The method according to claim 1, wherein the plurality of electrodes are configured to further ablate using at least one of a radiofrequency current and irreversible electroporation.

7. The method according to claim 1, wherein the catheter comprises a lasso catheter.

8. The method according to claim 1, wherein the indication comprises at least one of respective numerical values of distances between the plurality of the electrodes and the multiplicity of the locations and a color indicative of the values of the distances.

9. Apparatus, comprising:
a display device;
a catheter having a plurality of electrodes configured to ablate selected sites of an organ of a human subject; and
a processor, configured to:
register a multiplicity of locations of ablated sites of the organ;
record manipulations of the catheter in proximity to the organ;
render on the display device an organ icon showing the organ and the multiplicity of the locations of the ablated sites, and a catheter icon showing the plurality of electrodes; and
provide an indication on the display device of a quality of alignment between the plurality of electrodes and the multiplicity of the locations of the ablated sites.

10. The apparatus according to claim 9, and comprising the processor being configured to:
identify a gap in the locations, and, when the indication denotes there is good alignment,
record further manipulations of the catheter so that at least one of the plurality of electrodes aligns with a section of the gap, and
activate the at least one electrode to ablate the section.

11. The apparatus according to claim 10, wherein the multiplicity of the locations of the ablated sites define an ablation site plane, and the plurality of the electrodes define an electrode plane, so that a position and an orientation of the ablation site plane and the electrode plane correspond for the good alignment.

12. The apparatus according to claim 10, wherein identifying the gap comprises identifying from amongst the registered locations adjacent locations that are separated by greater than a preset distance.

13. The apparatus according to claim 10, wherein the organ comprises a pulmonary vein of the human subject, and wherein identifying the gap comprises fitting a closed curve to the locations of the ablated sites, and indicating on the display device at least one site of the closed curve wherein adjacent locations are separated by a distance greater than a preset threshold distance.

14. The apparatus according to claim 9, wherein the plurality of electrodes are configured to ablate using at least one of a radiofrequency current and irreversible electroporation.

15. The apparatus according to claim 9, wherein the catheter comprises a lasso catheter.

16. The apparatus according to claim 9, wherein the indication comprises at least one of respective numerical values of distances between the plurality of the electrodes and the multiplicity of the locations and a color indicative of the values of the distances.
